(19) United States
(12) Patent Application Publication
(10) Pub. No.: US 2013/0280270 A1
(43) Pub. Date: Oct. 24, 2013

(54) METHODS OF TREATING LUNG DISEASE AND OTHER INFLAMMATORY DISEASES

(71) Applicant: Duke University, Durham, NC (US)

(72) Inventors: Mary E. Sunday, Durham, NC (US); W. Michael Foster, Durham, NC (US); Shutang Zhou, Chapel Hill, NC (US); Erin Potts, Chapel Hill, NC (US)

(21) Appl. No.: 13/917,883

(22) Filed: Jun. 14, 2013

Related U.S. Application Data
(62) Division of application No. 13/201,501, filed on Oct. 12, 2011.
(60) Provisional application No. 61/157,445, filed on Mar. 4, 2009.

(51) Int. Cl. A61K 39/395; A61K 31/505

(52) U.S. Cl. CPC A61K 39/3955; A61K 31/505
USPC 424/152.1; 514/275

(57) ABSTRACT

A method of treating an inflammatory disease in a subject in need thereof is carried out by administering the subject a gastrin-releasing peptide (GRP) inhibitor in a treatment effective amount. Suitable GRP inhibitors include compounds of the general formula:

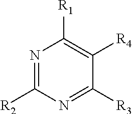

and pharmaceutically acceptable salt or prodrugs thereof.

### FIELD OF THE INVENTION

The present invention concerns methods of treating airway diseases and conditions such as asthma.

### BACKGROUND OF THE INVENTION

Asthma is a human disease of episodic airway obstruction, with the greatest resistance arising in the small airways where pulmonary neuroendocrine cells (PNECs) are concentrated. PNECs are epithelial cells that secrete bioactive neuropeptides including GRP, which act on neighboring cells in a paracrine fashion. Increased numbers of PNECs (PNEC hyperplasia) can develop in bronchioles in response to hypoxia, oxidant exposure and/or inflammation¹.

### SUMMARY OF THE INVENTION

A first aspect of the present invention is a method of treating an inflammatory disease in a subject in need thereof, comprising administering said subject a gastrin-releasing peptide (GRP) inhibitor in a treatment effective amount. In some embodiments, the administering step is carried out prophylactically or prior to the onset of inflammatory symptoms; in other embodiments, the administering step is carried out following the onset of an inflammatory symptoms.

A further aspect of the present invention is a method of treating an airway disease such as asthma in a subject in need thereof, comprising administering said subject a gastrin-releasing peptide (GRP) inhibitor in a treatment effective amount. In some embodiments, the administering step is carried out prophylactically or prior to the onset of an asthmatic response. In some embodiments, the administering step is carried out following the onset of an asthmatic response.

A further aspect of the invention is a method of treating an airway disease such as bronchiolitis obliterans in a subject in need thereof, comprising administering to said subject a gastrin-releasing peptide (GRP) inhibitor such as a small molecule gastrin-releasing peptide (GRP) inhibitor in a treatment effective amount. In some embodiments In some embodiments the subject is a lung transplant recipient, to treat chronic rejection. In some embodiments the subject is afflicted with a respiratory virus infection (e.g., respiratory syncytial virus).

A further aspect of the invention is a method of treating a lung parenchymal disease such as pulmonary fibrosis in a subject in need thereof, comprising administering to said subject a gastrin-releasing peptide (GRP) inhibitor such as a small molecule gastrin-releasing peptide (GRP) inhibitor in a treatment effective amount. In some embodiments, the subject is afflicted with idiopathic pulmonary fibrosis, other interstitial lung diseases, or radiation pneumonitis (In the latter case, the GRP inhibitor may be administered before or immediately following radiation exposure.).

A further aspect of the invention is a method of treating a lung parenchymal disease such as acute respiratory distress syndrome (ARDS) in a subject in need thereof, comprising administering to said subject a a gastrin-releasing peptide (GRP) inhibitor such as small molecule gastrin-releasing peptide (GRP) inhibitor in a treatment effective amount. In some embodiments the subject is an at-risk subject afflicted with sepsis, inhalational burn injury, or major trauma.

In some embodiments of the foregoing, the administering step is carried out by topically applying said GRP inhibitor to airway surfaces of said subject, such as by intratracheal instillation.

In some embodiments of the foregoing, the administering step is carried out by inhalation administration.

In some embodiments of the foregoing, the GRP inhibitor is a small molecule GRP inhibitor (e.g., a non-peptide GRP inhibitor).

In some embodiments of the foregoing, the GRP inhibitor is the monoclonal antibody 2A11, or an antibody that specifically binds to the eptiope bound by the monoclonal antibody 2A11 (or, a monoclonal antibody which specifically binds to a peptide having an amino-acid sequence identical to carboxy terminal heptapeptide region of bombesin and has the same antigen-binding specificity as antibodies produced by the deposited cell line having the American Type Culture Collection number HB8711, see, e.g., U.S. Pat. No. 5,109,115 to Cuttitta and Minna).

A further aspect of the invention is the use of a GRP inhibitor for carrying out a method as described herein above or below.

A further aspect of the invention is the use of a GRP inhibitor for the preparation of a medicament for carrying out a method as described herein above or below.

### BRIEF DESCRIPTION OF THE DRAWINGS

FIG. 1 summarizes cytokines and cell types involved in two in vivo mouse models of asthma, to ozone (air pollution) or ovalbumin (allergic airways inflammation)

FIG. 2A-2H show that the GRP inhibitor 77427 significantly suppresses (P<0.0001) or abrogates the increased levels of 21 of 21 cytokines tested (which represent at least 5 different inflammatory cell types) in the ozone and ovalbumin mouse models of asthma.

FIG. 2A shows suppression of Interleukin-2 (IL-2)

FIG. 2B shows suppression of IL-2 (p40)

FIG. 2C shows suppression of TNF-alpha.

FIG. 2D shows suppression of GM-CSF.

FIG. 2E shows suppression of IL-4.

FIG. 2F shows suppression of IL-5.

FIG. 2G shows suppression of IL-13.

FIG. 2H shows suppression of IL-6.

FIG. 2I shows suppression of IL-17.

FIG. 2J shows suppression of MCP-1.

FIG. 2K shows suppression of KC.

FIG. 2L shows suppression of VEGF.

### DETAILED DESCRIPTION OF PREFERRED EMBODIMENTS

The present invention is primarily concerned with the treatment of human subjects, but the invention may also be carried out on animal subjects, particularly mammalian subjects such as dogs, cats, livestock and horses for veterinary purposes. Subjects may be male or female and may be of any age, including neonate, infant, juvenile, adolescent, adult, or geriatric subjects.

“Treat” as used herein refers to any type of treatment that imparts a benefit to an aging patient, particularly delaying or retarding the progression of aging.

“Pharmaceutically acceptable” as used herein means that the compound or composition is suitable for administration to a subject to achieve the treatments described herein, without unduly deleterious side effects in light of the severity of the disease and necessity of the treatment.

“Pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

“Administering” as used herein may be by any suitable route of administration, including but not limited to topical, oral, parenteral (e.g., subcutaneous, intravenous, intramuscular, and intraperitoneal injection, etc.), and by inhalation administration (e.g., topical application to airway surfaces).

“Antibody” as used herein refers to all types of immunoglobulins, including IgG, IgM, IgA, IgD, and IgE. Of these, IgM and IgG are particularly preferred. The antibodies may be monoclonal or polyclonal and may be of any species of origin, including (for example) mouse, rat, rabbit, horse, or human, or may be chimeric antibodies. See, e.g., M. Walker et al., Molec. Immunol. 26, 403-11 (1989). The antibodies may be recombinant monoclonal antibodies produced according to the methods disclosed in Reading U.S. Pat. No. 4,474,893, or Cabilly et al., U.S. Pat. No. 4,816,567. The antibodies may also be chemically constructed by specific antibodies made according to the method disclosed in SegAl et al., U.S. Pat. No. 4,676,980.

“Inflammatory disorder” or “inflammatory disease, as used herein, includes, but is not limited to, inflammatory bowel disease; contact sensitivity, eczematous dermatitis or other types of inflammatory dermatitis; atherosclerosis; temporal arteritis; autoimmune diseases including systemic lupus erythematosis, rheumatoid arthritis (RA), multiple sclerosis (MS) and some cases of diabetes mellitus; viral hepatitis, degenerative joint disease; and Alzheimer's disease

### 1. Active Compounds.

Active compounds useful for carrying out the present invention include peptide gastrin-releasing peptide (GRP) inhibitors and non-peptide or small molecule GRP inhibitors. Numerous such compounds are known. Examples include, but are not limited to, those described in R. Jensen et al., Pharmacological Reviews, 60, 1-42 (2008) and U.S. Pat. Nos. 5,047,502; 5,019,647; 5,109,115; 5,244,883; 5,460,801; 5,620,955; 5,620,959; 5,834,433; 6,194,437; 6,989,371; 7,147,838; and US Patent Application Publication No. 2008/0090758 (published Apr. 17, 2008). The disclosures of all United States patent references cited herein are incorporated by reference herein in their entirety.

Active compounds useful for carrying out the present invention include but are not limited to those small molecule GRP inhibitors described in F. Cuttitta et al., US Patent Application Publication No. 2008/0249115 (published Oct. 9, 2008). Particular examples of compounds described therein, and which may be used to carry out the present invention, include but are not limited to compounds of the formula:

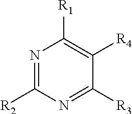

wherein:

R₁ is —R₅—(CH₂)ₙ—CH(R₆)OH, and R₅ is NH, S or O, R₆ is H or CH₃, and n is an integer from 1-4;

R₂ is NH₂, substituted amino or acetamide;

R₃ is H, halogen, CH₃, or CF₃; and

R₄ is H, alkyl, substituted alkyl, alkenyl, alkoxy or halogen;

or a pharmaceutically acceptable salt or prodrug thereof. A particular example is a compound of the formula:

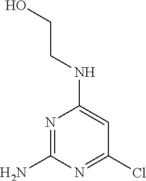 [SMILES: Nc1nc(Cl)cc(NCCO)n1]

or a pharmaceutically acceptable salt or prodrug thereof.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### 2. Antibody Active Compounds.

The monoclonal antibody 2A11 or monoclonal antibodies produced by the deposited cell line having the American Type Culture Collection number HB8711, are useful for carrying out the present invention, and are known and described in U.S. Pat. No. 5,109,115 to Cuttitta and Minna, the disclosure of which is incorporated herein by reference.

Antibodies that specifically bind to the epitope (or “target epitope”) bound by monoclonal antibodies produced by the deposited cell line having the ATCC No. HB8711 ((i.e., antibodies which bind to a single antigenic site or epitope on the protein) are useful for a variety of diagnostic and therapeutic purposes. Antibodies to the target epitope may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

Antibody fragments which contain specific binding sites for the target epitope may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., Science 254, 1275-1281 (1989)).

For the production of antibodies, various hosts including goats, rabbits, rats, mice, humans, and others, may be immunized by injection with the target epitope or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Monoclonal antibodies to the target epitope may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J Immunol, Methods 81:31-42; Cote, R. J. et cd. (1983) Proc. Natl. Acad. Sci. 80:2026-2030; Cole, S. P. et al. (1984) Mol. Cell. Biol. 62:109-120). Briefly, the procedure is as follows: an animal is immunized with the target epitope or immunogenic fragments or conjugates thereof. For example, haptenic oligopeptides of the target epitope can be conjugated to a carrier protein to be used as an immunogen. Lymphoid cells (e.g. splenic lymphocytes) are then obtained from the immunized animal and fused with immortalizing cells (e.g. myeloma or heteromyeloma) to produce hybrid cells. The hybrid cells are screened to identify those which produce the desired antibody.

Human hybridomas which secrete human antibody can be produced by the Kohler and Milstein technique. Although human antibodies are especially preferred for treatment of human, in general, the generation of stable human-human hybridomas for long-term production of human monoclonal antibody can be difficult. Hybridoma production in rodents, especially mouse, is a very well established procedure and thus, stable murine hybridomas provide an unlimited source of antibody of select characteristics. As an alternative to human antibodies, the mouse antibodies can be converted to chimeric murine/human antibodies by genetic engineering techniques. See V. T. Oi et al., Bio Techniques 4(4):214-221 (1986); L. K. Sun et al., Hybridoma 5 (1986).

In addition, techniques developed for the production of “chimeric antibodies”, the splicing of mouse antibody genes to human antibody genes to obtain a molecule with appropriate antigen specificity and biological activity can be used (S. L. Morrison, et al. Proc. Natl. Acad. Sci. 81, 6851-6855 (1984); M. S. Neuberger et al., Nature 312:604-608 (1984); S. Takeda, S. et al., Nature 314:452-454 (1985)). Alternatively, techniques described for the production of single chain antibodies may be adapted, using methods known in the art, to produce target-specific single chain antibodies. Antibodies with related specificity, but of distinct idiotypic composition, may be generated by chain shuffling from random combinatorial immunoglobin libraries (D. R. Burton, Proc. Natl. Acad. Sci. 88, 11120-3 (1991)).

Antibodies may also be produced by inducing in vivo production in the lymphocyte population or by screening immunoglobulin libraries or panels of highly specific binding reagents as disclosed in the literature (R. Orlandi et al., Proc. Natl. Acad. Sci. 86, 3833-3837 (1989)); G. Winter et al., Nature 349, 293-299 (1991)).

### 3. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces such as by aerosol administration as described, for example, in U.S. Pat. No. 4,501,729) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The active and supplemental compounds described herein may be administered to the lungs of a patient by any suitable means, but are preferably administered via an aerosol suspension of respirable particles comprised of the active compound, which the subject inhales. The active compound can be aerosolized in a variety of forms, such as, but not limited to, dry powder inhalants, metered dose inhalants, or liquid/liquid suspensions. The respirable particles may be liquid or solid.

The particulate pharmaceutical composition may optionally be combined with a carrier to aid in dispersion or transport. A suitable carrier such as a sugar (i.e., lactose, sucrose, trehalose, mannitol) may be blended with the active compound or compounds in any suitable ratio (e.g., a 1 to 1 ratio by weight).

Solid or liquid particulate forms of the active compound prepared for practicing the present invention should include particles of respirable size: that is, particles of a size sufficiently small to pass through the mouth and larynx upon inhalation and into the bronchi and alveoli of the lungs. In general, particles ranging from about 1 to 10 microns in size are within the respirable range. Particles of non-respirable size which are included in the aerosol tend to be deposited in the throat and swallowed, and the quantity of non-respirable particles in the aerosol is preferably minimized.

In the manufacture of a formulation according to the invention, active compounds of the present invention or the pharmaceutically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation which may contain from 0.5% to 99% by weight of the active compound. One or more active compounds may be incorporated in the formulations of the invention, which formulations may be prepared by any of the well-known techniques of pharmacy consisting essentially of admixing the components.

Aerosols of liquid particles comprising the active compound may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water or normal saline or phosphate-buffered saline [PBS] (and most preferably sterile, pyrogen-free water or normal saline or PBS) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 200 .mu.l, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

Any propellant may be used in carrying out the present invention, including both chlorofluorocarbon-containing propellants and non-chlorofluorocarbon-containing propellants. Thus, fluorocarbon aerosol propellants that may be employed in carrying out the present invention including fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to, those described in U.S. Pat. No. 6,451,288.

Compositions containing respirable dry particles of micronized active compound of the present invention may be prepared by grinding the dry active compound with, e.g., a mortar and pestle or other appropriate grinding device, and then passing the micronized composition through a 400 mesh screen to break up or separate out large agglomerates.

The aerosol, whether formed from solid or liquid particles, may be produced by the aerosol generator at a rate of from about 10 to 150 liters per minute. Aerosols containing greater amounts of medicament may be administered more rapidly. Typically, each aerosol may be delivered to the patient for a period from about 30 seconds to about 20 minutes, with a delivery period of about one to ten minutes being preferred.

### 4. Dosage and Routes of Administration.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration, or topically to the airway surfaces or lungs of the subject, such as by aerosol inhalation.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. Typically, a dosage from about 0.5 mg/kg to 5 mg/kg may be employed for intramuscular injection.

The present invention is explained in greater detail in the following non-limiting Examples.

### Example 1

Without wishing to be bound by any particular theory of the invention, the central hypothesis of this invention, as currently understood, is that GRP overproduction by PNECs plays a role in the pathophysiology of both acute and chronic phases of asthma, as well as other inflammatory lung diseases including bronchiolitis and interstitial fibrosis. This hypothesis is supported by cumulative observations, which are presented below.

We demonstrated that GRP gene expression is increased in the lung 24 hours after birth in the most clinically relevant baboon model of chronic lung disease of newborns (also called bronchopulmonary dysplasia, or BPD)²⁻⁴. GRP and its amphibian homologue bombesin are potent, immediate bronchoconstrictors in vitro^{5,6}. The presence of GRP in the lung was not reported until 1978⁷. PNEC hyperplasia was observed in guinea pigs given systemic antigen sensitization, in which PNEC degranulation follows aerosol challenge⁸. To date, there are no reports of PNEC hyperplasia in human asthmatics, and GRP has never been implicated directly in the pathophysiology of inflammatory airways disease in patients or in animal models. Suggestive evidence is derived from our studies of BPD, in which we showed that GRP is a pro-inflammatory mediator of lung injury⁴. GRP levels are elevated shortly after birth in newborn baboons and premature human infants that later develop BPD, and GRP blockade abrogates lung injury in both baboon models of BPD^{4,9}, We showed that GRP triggers mast cell proliferation and chemotaxis¹⁰, eosinophil chemotaxis¹¹, T cell migration (M. Sunday, unpublished data), and autoimmune T cell responses¹², High-affinity GRP receptors are present in mesenchymal cells surrounding airways³, where remodeling occurs in patients with chronic asthma¹³. GRP is also chemotactic for macrophages¹⁴ and fibroblasts¹⁵, and induces pulmonary fibroblast proliferation¹¹⁶. All of these cell types have been implicated in the pathogenesis of acute and/or chronic asthma^{17,18}. Our observation that GRP mediates lung injury in BPD is relevant to asthma because BPD patients are 5-10-fold more likely to develop pediatric asthma^{19,20}.

Asthma is characterized by intermittent airways obstruction with inflammation and broncho-constriction involving both large (cartilaginous) airways and bronchioles. There is evidence that bronchioles are the most critical site for airway narrowing because the greatest airflow resistance (R) occurs in small caliber tubes (R ˜1/radius). Also, there are ˜10-fold more bronchioles than cartilaginous airways. Asthmatic responses can be triggered by allergens, pollution, irritants, or infections, leading to permanent structural remodeling with wall thickening²¹. The typical inflammatory cells involved in asthmatic responses are mast cells, eosinophils, and Th2 lymphocytes, representing innate and adaptive immunity²². It is widely believed that mast cells are important for both acute exacerbations and airway remodeling in asthma. Many cytokines and mediators derived from these cells modulate inflammation, especially during the effector phase²³. A few studies have analyzed the inflammatory role of neuropeptides from nerve fibers, termed “neurogenic inflammation”, with a focus on substance P, neurokinins, and calcitonin gene-related peptide²⁴. In spite of this large body of information, it remains unclear why asthma is increasing in incidence worldwide, now afflicting >15 million adults and children in the USA, where it causes 4,500 deaths annually and is the #1 pediatric healthcare burden. Despite optimal medical management, many asthmatics are treatment-resistant and/or have progressive disease²⁵. For example, in a subset of patients, beta-agonists paradoxically led to a clinical decline²⁶. Such diversity in treatment responses and disease progression is attributed to interactions between environmental factors and multi-genetic variability. Clinical efforts to prevent asthma have been disappointing, with only modest effects in all age groups^{27,28}. Over 35 genes linked to asthma have been identified²³, but few are related to known asthma mediators. Despite the wealth of knowledge about cells and inflammatory mediators implicated in asthma, no agent has been identified that can effectively prevent and/or treat this debilitating condition that is reaching epidemic proportions.

A major trigger of asthmatic responses in children and older adults is viral infection of the lower respiratory tract, most often by respiratory syncytial virus (RSV) or rhinovirus (RV), leading to bronchiolitis and/or pneumonia^{29,30}, Bronchiolitis alone accounts for the largest number of pediatric hospital admissions: 120,000 patients admitted per year in the USA, 80,000 of which are due to RSV. Later, these infants have a 2-3-fold increased risk of asthma at 5-10 years of age, which is further exacerbated by exposure to environmental tobacco smoke. It is not known whether viral bronchiolitis precipitates asthma, or whether infants with a genetic predisposition to asthma are more prone to developing bronchiolitis²⁹.

PNEC Hyperplasia and GRP Over-Production: Clinical Associations and Animal Models.

PNEC hyperplasia is associated with many inflammatory conditions of the lung, including BPD³¹, cystic fibrosis³², and chronic obstructive pulmonary disease (COPD)^{33,34}. Asymptomatic smokers have elevated GRP levels in their bronchoalveolar lavage (BAL) fluid and in their urine^{35,36}; smokers have a markedly elevated risk of developing asthma³⁷. Adults with primary idiopathic PNEC hyperplasia have secondary increased airways reactivity³⁸. Children with primary idiopathic PNEC hyperplasia have air trapping and elevated airways reactivity³⁹. Conversely, we hypothesized that pro-inflammatory cytokines induce increased numbers of PNECs in the bronchioles. Testing this, we showed that TNFa can induce PNEC differentiation¹, with rapid induction of neuroendocrine (NE)-specific gene expression followed by a delayed phenotypic shift. PNEC hyperplasia is observed in guinea pigs given systemic antigen sensitization, in which PNEC degranulation follows aerosol challenge⁸. At present, there are no reports of PNEC hyperplasia in human asthmatics, and GRP has never been implicated directly in the pathophysiology of inflammatory airways disease in patients or in animal models.

Bombesin is a 14-amino acid peptide that was first identified in frog skin⁴⁰. GRP is a mammalian homolog of bombesin⁴¹. Bombesin and GRP have the same bioactive peptide sequence and both act at the same high-affinity GRP receptor that is expressed in parallel with GRP in developing lung⁴². GRP is normally expressed at highest levels in mid-gestation human fetal lung, in which it plays an important role in promoting normal lung development⁴³.

We have strong evidence that GRP is a pro-inflammatory mediator of lung injury in BPD⁴. Increased numbers of GRP-positive PNECs occur in the lungs of human infants dying with BPD³¹. We began to test the hypothesis that GRP causes BPD using two different approaches. In two baboon models (100% hyperoxia versus barotrauma) and in premature human infants, we demonstrated that GRP levels are elevated in urine shortly after birth only in animals that develop BPD, but long before there are clinical or pathological manifestation of BPD⁴. These urine GRP levels reflect increased GRP gene expression in the lung right after birth, even before there are increased inflammatory cytokines². Over 90% of urine GRP is derived from the lung⁴. We measure GRP levels in urine because urine is freely available from individual animals, permitting kinetic analyses. Furthermore, BLP is stable and concentrated in the urine.

To determine the clinical relevance of these findings, we analyzed urine samples from 132 human infants born at <28 weeks gestation during the first 5 days after birth⁹. A single elevated BLP level during the first 5 postnatal days was associated with a tenfold increased risk of developing BPD 2-3 months later (P<0.001). In contrast, two known major risk factors for BPD, mechanical ventilation and prematurity, each conferred only a 2-3-fold increased risk of BPD (P=0.03 and 0.10, respectively). Therefore, elevated urine BLP shortly after birth is by far the strongest predictor of BPD occurring much later in human infants.

Next, we treated premature baboons from both models with a blocking anti-GRP antibody, and showed that this GRP blockade abrogates lung injury^{4,9}. We began to explore mechanisms for this protective effect. In both baboon BPD models, GRP overproduction is prevented by antioxidant treatment⁴⁴ (and M. sunday and S. Andreeva, unpublished data), indicating that GRP elevation is partly due to free-radical injury. Downstream, GRP triggers mast cell proliferation and chemotaxis¹⁰, eosinophil chemotaxis, T cell migration and autoimmune T cell responses¹². GRPR is expressed at high levels in mesenchymal cells around airways³, where remodeling and fibrosis occur. GRP is known to be chemotactic for macrophages⁴⁵ and fibroblasts¹⁵, and induces fibroblast proliferation¹. These cell types have all been implicated in the pathogenesis of asthma and fibrotic lung diseases⁴⁶. Our observation that GRP mediates lung injury in early BPD is relevant to asthma because about half of BPD patients develop pediatric asthma^{19,20}.

GRP also alters adaptive immunity in baboons with BPD¹². We observed peripheral immunodeficiency (lack of PHA responsiveness) and increased anti-lung reactivity in BPD, both of which were reversed by GRP blockade. Numerous CD4+ cells are present in the interstitium of BPD animals, but not in non-BPD controls. A role for adaptive immunity in the pathogenesis of BPD represents a paradigm shift in our understanding of this chronic lung disease.

Increased PNECs occur in animal models of allergic airways inflammation, in which they could play an important role in regulating innate immunity. Classical inflammatory responses in asthma include both the innate and adaptive immune systems, with activation of mast cells, macrophages, and T cells by allergens, irritants, smoke, and other agents. We propose that asthmatic patients develop PNEC hyperplasia as part of their innate immune response, with secretion of distinct bioactive peptides leading to symptoms that might not respond to conventional treatment. There is evidence that genes could predispose individuals to developing PNEC hyperplasia. Some inbred strains of rats are resistant to developing PNEC hyperplasia in response to hypoxia⁴³. PNEC hyperplasia can be induced in mice over-expressing v-Ha-ras in PNECs⁴⁷, rats exposed to cigarette smoke⁴⁸, monkeys treated with nicotine⁴⁹, and hamsters given hyperoxia with the tobacco-specific nitrosamine, NNK. Conversely, cigarette smoke can lead to exacerbations of asthma. Thus, we hypothesize that GRP could contribute to multiple subtypes of asthma or other inflammatory or fibrotic lung diseases⁵⁰.

We hypothesize that asthmatic patients are genetically predisposed to overproducing GRP, which in turn leads to asthma. Possible genetic mechanisms include: 1. Decreased GRP promoter methylation leading to GRP overexpression; 2. Altered expression of genes that promote PNEC hyperplasia and increased GRP, such as Notch; 3. Elevated GRP levels due to decreased levels of enzymes that degrade GRP; and 4. GRP promoter SNPs (single nucleotide polymorphisms) that alter the binding of transcription factors regulating GRP gene expression.

Another condition associated with asthma⁵¹ is gastro-esophageal reflux (GER), with exacerbation of lung symptoms apparently caused by acid aspiration. My novel, unconventional hypothesis is that GER may be due to GRP overproduction leading to increased gastrin and thus elevated acid production. If this is true, when GRP blockade is ultimately tested in asthmatics, this should reduce GER as well as airways resistance. GRP blockade could also be useful for treating GER.

Prior Art.

Two prior publications in 1973 and 1993^{5,6} demonstrated that bombesin or GRP treatment in vitro can cause smooth muscle constriction of guinea pig tracheal smooth muscle. It was determined that this is likely mediated via the high-affinity GRP receptor, One in vitro experiment was reported using a GRP receptor antagonist ([Leu¹³-(ψ-CH₂NH)-Leu¹⁴]-bombesin) in lung slices from one non-human primate⁵²: 2 slices were treated with methacholine (MCh) alone and 2 slices with MCh+[Leu¹³-(ψ-CH₂NH)-Leu¹⁴]-bombesin. Based on morphological assessment of airway diameter, the bronchi but not the bronchioles treated with MCh had reduced internal airway diameter consistent with bronchoconstriction that was slightly reduced by the GRP receptor antagonist. The limitations of the latter approach is that only modest changes occurred in airway reactivity of large airways, whereas asthma in humans is primarily a disease of the small airways, Furthermore, GRP is the only bioactive peptide known to be expressed prior to the development of lung injury and inflammation^{2,4,9}, and as such is an early biomarker of inflammatory lung disease.

### Preferred Embodiments

A currently preferred way to carry out the invention is to use the small molecule GRP blocking agent, 77427. Optimally, 77427 should be used as an aerosol, which would deliver the agent in highest concentrations to the lung, its site of action. Stocks of 77427 are prepared at high concentrations in dimethylsulfoxide (DMSO) [2 to 50 mg/ml=0.6 to 14.5 mM]. The working concentrations of 77427 range from 0.1 up to 500 nM, made up by diluting the DMSO stock in phosphate-buffered saline. As a guideline, 500 nM blocks 95% of GRP, 50 nM blocks 70% of GRP, and 5 nM blocks 50% of GRP⁵³,

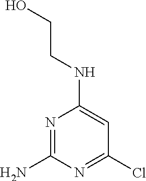 [SMILES: Nc1nc(Cl)cc(NCCO)n1]

77427 was developed as a potential agent for treating cancer by Frank Cuttitta at the National Cancer Institute (NCI) using the Developmental Therapeutics Program (DTP)'s small molecule “Diversity Set” of compounds, a collection of 2,000 family members representing over 500,000 candidate drugs in the DTP combinatorial library⁵⁴. Neutralizing monoclonal antibody to GRP, MoAb-2A11⁵⁵, was labeled with peroxidase and used as the detection agent to identify small molecule drugs capable of binding solid phased GRP and thus preventing antibody-ligand interaction, Candidate molecules making it through this first level screen were then assessed for their effectiveness in blocking GRP-induced biological events, inositol 1,4,5-triphosphate (IP3) production and Ca²⁺ flux. One such compound, NSC 77427, proved to be an ideal mimetic to MoAb-2A11 and was selected for further analysis as an antagonist to GRP. It appears that 77427 has a long half-life of ˜10 days (F. Cuttitta, unpublished data).

This compound (77427) is a potent inhibitor of GRP-induced angiogenesis in vitro and in vivo⁵³. It is an effective blocker of GRP mediated endothelial cell tube formation on Matrigel support matrix and on a molar basis (≈500 nM), gives an equivalent suppressive response as MoAb-2A11 in stopping neovascularization in the nude mouse directed in vivo angiogenesis assay. An NCI patent has been issued for NSC 77427 as a MoAb-2A11 mimetic and a suppressor drug preventing GRP regulated biological events (60/500,650 and 60/569,625). The NIH Angiogenesis Core suggests that for “Proof-of-Principle” the MoAb2A11 should be tested to validate that biological neutralization of GRP is responsible for any observed biological effects, such as suppression of airways hyperreactivity in test animals. In addition, we could consider substituting additional small molecule mimetics to MoAb-2A11 as alternative therapeutic drugs (NSC 54671 and NSC 112200. Thus far, the introductory route of NSC 77427 in our animal studies has been via intraperitoneal injection, but addition of 77427 to the supply water or aerosols are possible other avenues to consider.

### Examples

We have carried out experiments with GRP blockade given as an intraperitoneal (IP) injection to prevent and/or treat AHR in several different mouse models of airways hyperreactivity (AHR) and/or inflammation: ozone-induced AHR as a model for asthma precipitated by air pollution, ovalbumin (OVA)-induced AHR as a model of allergic airways inflammation, and endotoxin (lipopolysaccharide, LPS)-induced AHR as a first step towards assessing infectious causes of asthma. These experiments used the small molecule GRP inhibitor, 77427, or a monoclonal anti-GRP blocking antibody, 2A 11. The invention would extend to any compound able to block GRP signaling by either binding to GRP itself or to the GRP receptor, which are part of the same signal transduction cascade. In the near future, we hope to test GRP blockade in additional mouse models of asthma, including: Mycoplasma infection, respiratory syncytial virus (RSV) infection, Aspergillus infection, and house dust mite exposure.

We can also block chronic radiation pneumonitis in mice given 500 nM 77427 twice a week IP following 15 Gy thoracic radiation.

### Example

### Cytokines Suppressed in Mouse Models of Asthma

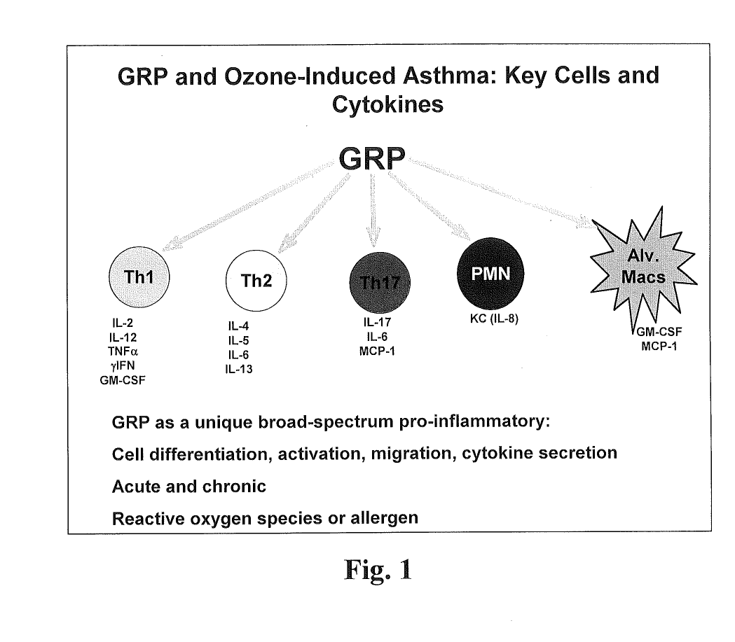

Using 2 in vivo mouse models of asthma, to ozone (air pollution) or ovalbumin (allergic airways inflammation), we have demonstrated that 77427 significantly suppresses (P<0.0001) or abrogates the increased levels of 21 of 21 cytokines tested, which represent at least 5 different inflammatory cell types. These cytokines and cell types are summarized as shown in FIG. 1.

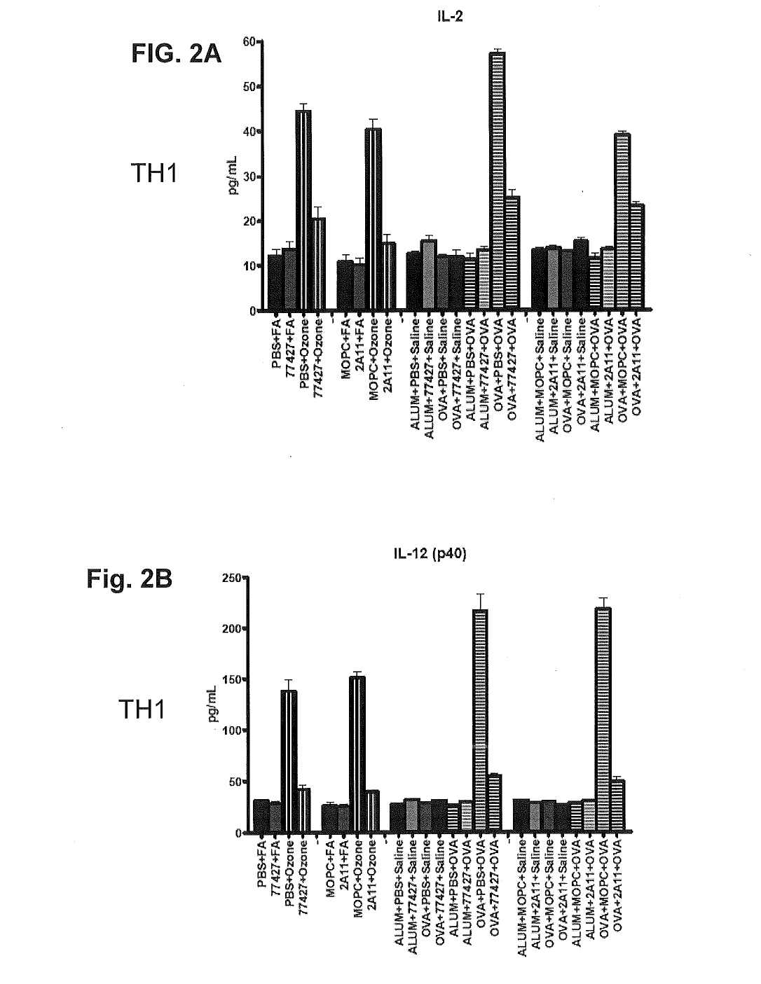

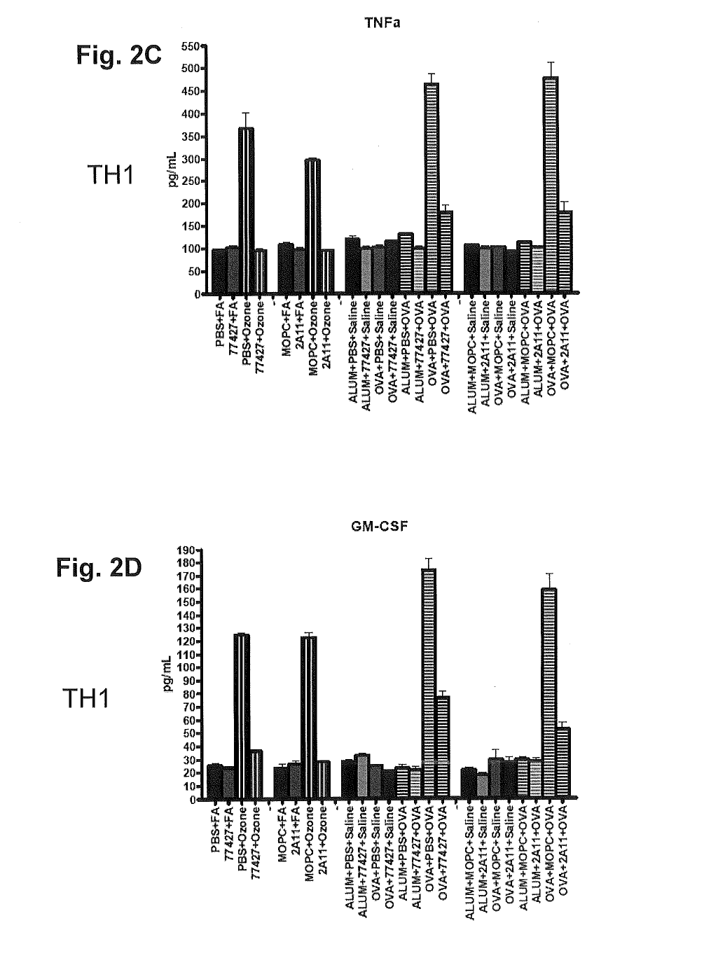

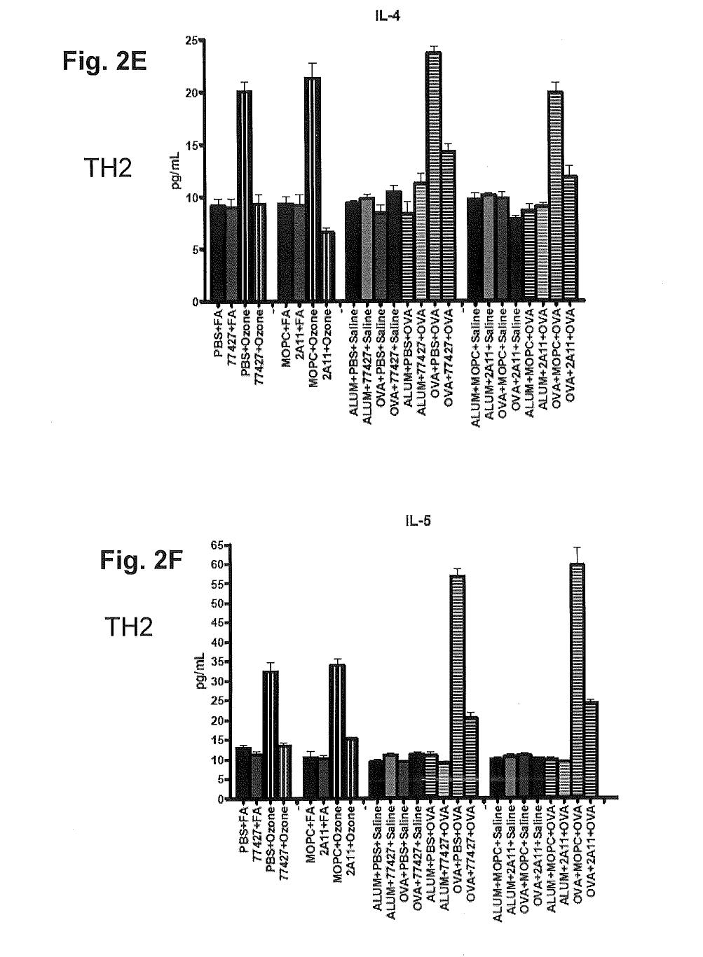

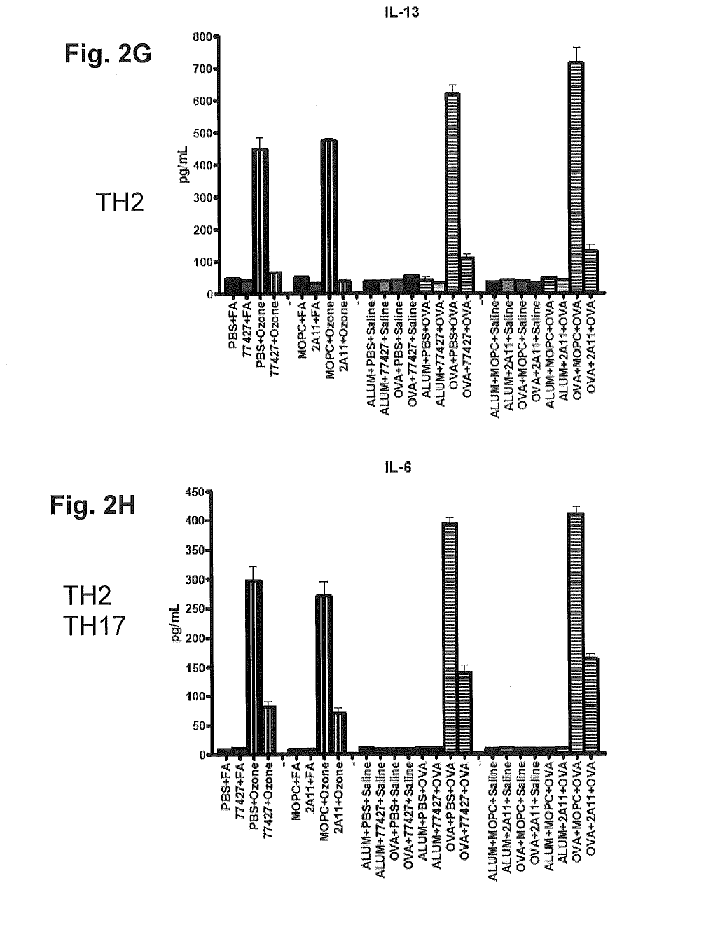

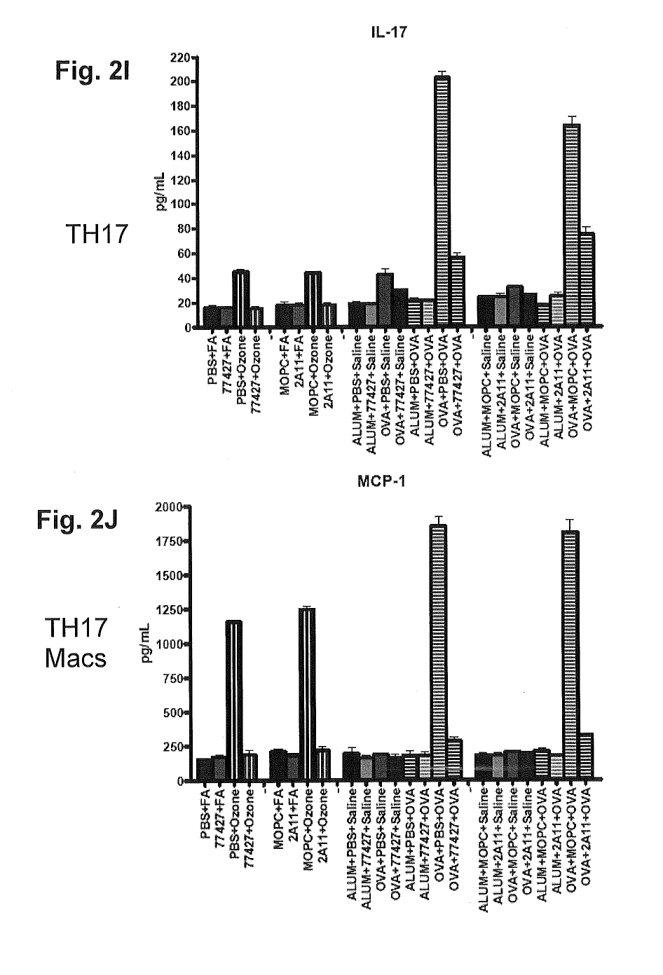

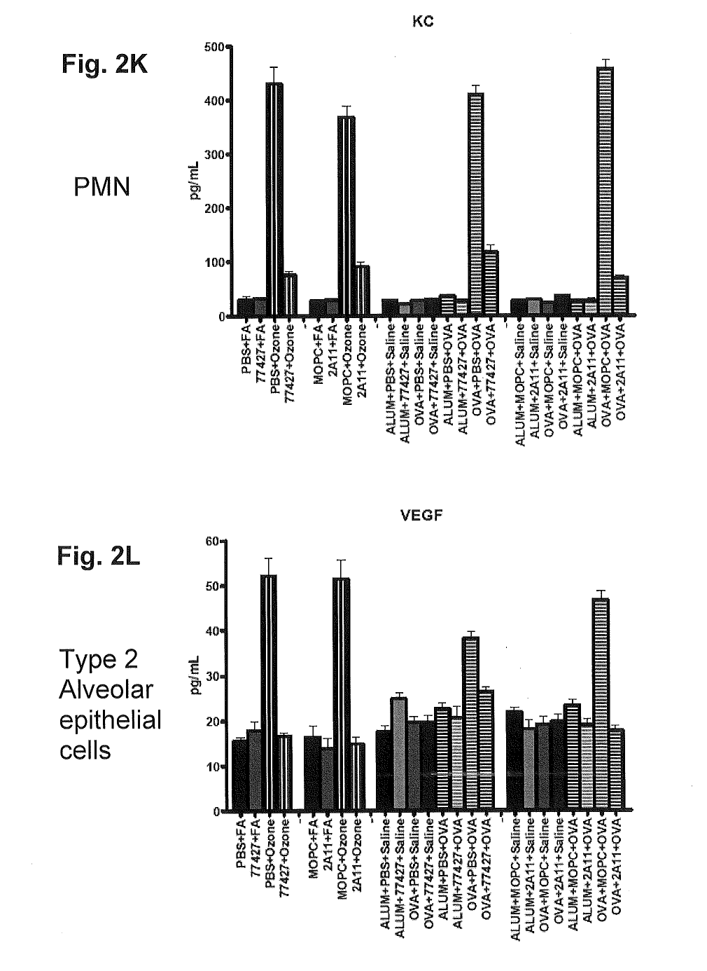

Additional details of cytokines decreased by 77427 are given in the FIGS. 2A-2L. With regards to airways inflammation, in both the ozone and OVA models 77427 abrogates both airways inflammation and airway hyperreactivity (AHR). As a specificity control, the GRP blocking antibody 2A11 was used, giving the same results as 77427. In contrast, mice were treated with dexamethasone (Dex), the standard of care for asthma, before ozone exposure. Dex did not alter AHR or airways inflammation, and significantly suppressed only 4 of 21 cytokines (IL-9, IL-17, VEGF, RANTES). Moreover, Dex treatment before ozone resulted in significantly increased levels of 3 cytokines (IL-6, IL-12 (p40), TNFα).

### Summary of Cytokines Decreased by 77427

### Th1: IL-2, IL-12, TNFα, γIFN, GM-CSF

### Th2: IL-4, IL-5, IL-6, IL-13

### Th17: IL-17, IL-6, MCP-1

### PMN: KC (IL-8)

### AMs: GM-CSF, MCP-1

### Epith: VEGF

### REFERENCES

1. Sunday, M. E., Shan, L. & Subramaniam, M. Immunomodulatory functions of the diffuse neuroendocrine system: implications for bronchopulmonary dysplasia. Endocr Pathol 15, 91-106. (2004).
2. Subramaniam, M. et al. Bombesin-like peptides modulate alveolarization and angiogenesis in bronchopulmonary dysplasia. Am J Respir Crit. Care Med 176, 902-12 (2007).
3. Cullen, A. et al. Bombesin-like peptide (BLP) and BLP receptors in two different baboon models of bronchopulmonary dysplasia: diversity in gene expression yet similarity in function. Peptides 2000; 21:1627-1638. Peptides 21, 1627-1638 (2000),
4. Sunday, M. E., Yoder, B. A., Cuttitta, F., Haley, K. J. & Emanuel, R. L. Bombesin-like peptide mediates lung injury in a baboon model of bronchopulmonary dysplasia. J. Clin. Invest. 102, 584-594 (1998).
5. Impicciatore, M. & Bertaccini, G. The bronchoconstrictor action of the tetradecapeptide bombesin in the guinea-pig. Journal of Pharmacy and Pharmacology 25, 872-875 (1973).
6. Lach, E., Haddad, E. B. & Gies, J. P. Contractile effect of bombesin on guinea pig lung in vitro: involvement of gastrin-releasing peptide-preferring receptors. Am J Physiol 264, L80-6. (1993).
7. Wharton, J. et al. Bombesin-like immunoreactivity in the lung. Nature 273, 769-770 (1978).
8. Bousbaa, H. & Fleury-Feith, J. Effects of a long-standing challenge on pulmonary neuroendocrine cells of actively sensitized guinea pigs. Am. Rev. Respir. Dis. 144, 714-717 (1991).
9. Cullen, A., Van Matter, L. J., Moore, M., Parad, R. & Sunday, M. E. Urine bombesin-like peptide elevation precedes clinical evidence of bronchopulmonary dysplasia. Am. J. Respir. Crit. Care Med 165, 1093-1097 (2002).
10. Subramaniam, M. et al. Bombesin-like peptides and mast cell responses: relevance to bronchopulmonary dysplasia? Am J Respir Crit. Care Med 168, 601-11 (2003).
11. Subramaniam, M., Sugiyama, K., Coy, D. H., Weller, P. F. & Sunday, M. E. Bombesin-like peptides induce eosinophil chemotaxis via specific receptors. Am. J. Respir. Crit. Care Med. (abstract) (2002).
12. Rosen, D. M., Lee, J. H., Cuttitta, F., Rafigi, F. & Sunday, M. E. Accelerated thymic maturation and autoreactive T-cells in bronchopulmonary dysplasia. Am J Respir Crit. Care Med 174, 75-83 (2006).
13. Miller, M. et al. Corticosteroids prevent myofibroblast accumulation and airway remodeling in mice. Am J Physiol Lung Cell Mol Physiol 290, L162-9. (2006).
14. Del Rio, M. & De la Fuente, M. Chemoattractrant capacity of bombesin, gastrin-releasing peptide and neuromedin C is mediated through PKC activation in murine peritoneal leucocytes. Peptides 49, 185-193 (1994).
15. Yule, K. A. & White, S. R. Migration of 3T3 and lung fibroblasts in response to calcitonin gene-related peptide and bombesin. Exp Lung Res 25, 261-73. (1999).
16. Aguayo, S. M. et al. Increased pulmonary neuroendocrine cells with bombesin-like immunoreactivity in adult patients with eosinophilic granuloma. J Clin Invest 86, 838-44. (1990).
17. Bradding, P., Walls, A. F. & Holgate, S. T. The role of the mast cell in the pathophysiology of asthma. J Allergy Clin Immunol 117, 1277-84. Epub 2006 Apr. 27. (2006).
18. Galli, S. J. et al. Mast cells as “tunable” effector and immunoregulatory cells: recent advances. Annu Rev Immunol 23, 749-86. (2005).
19. Ng, D. K., Lau, W. Y. & Lee, S. L. Pulmonary sequelae in long-term survivors of bronchopulmonary dysplasia. Pediatr. Int. 42, 603-607 (2000).
20. Evans, M., Palta, M., Sadek, M., Weinstein, M. R. & Peters, M. E. Associations between family history of asthma, bronchopulmonary dysplasia, and childhood asthma in very low birth weight children. American Journal of Epidemiology 148, 460-466 (1998).
21. Bai, T. R. & Knight, D. A. Structural changes in the airways in asthma: observations and consequences. Clin Sci (Load) 108, 463-77. (2005).
22. Hamid, Q., Tulic, M. K., Liu, M. C. & Moqbel, R. Inflammatory cells in asthma: mechanisms and implications for therapy. J Allergy Clin Immunol 111, S5-S12; discussion S12-7. (2003).
23. Lilly, C. M. Diversity of asthma: evolving concepts of pathophysiology and lessons from genetics. J Allergy Clin Immunol 115, S526-31. (2005).
24. Groneberg, D. A., Quarcoo, D., Frossard, N. & Fischer, A. Neurogenic mechanisms in bronchial inflammatory diseases. Allergy 59, 1139-52. (2004).
25. Gaga, M. et al. Risk factors and characteristics associated with severe and difficult to treat asthma phenotype: an analysis of the ENFUMOSA group of patients based on the ECRHS questionnaire. Clin Exp Allergy 35, 954-9. (2005).
26. Shore, S. A. & Drazen, J. M. Beta-agonists and asthma: too much of a good thing? J Clin Invest 112, 495-7. (2003).
27. Becker, A. & Chan-Yeung, M. Primary asthma prevention: is it possible? Curr Allergy Asthma Rep 8, 255-61. (2008).
28. Chan-Yeung, M. & Becker, A. Primary prevention of childhood asthma and allergic disorders. Curr Opin Allergy Clin Immunol 6, 146-51, (2006).
29. Singh, A. M., Moore, P. E., Gem, J. E., Lemanske, R. F., Jr. & Hartert, T. V.
Bronchiolitis to asthma: a review and call for studies of gene-virus interactions in asthma causation. Am J Respir Crit. Care Med 175, 108-19 (2007).
30. (CDC), C.f.D.C.a.P. Brief report: respiratory syncytial virus activity—United States, 2005-2006. MMWR Morb Mortal Wkly Rep 55, 1277-9. (2006).
31. Johnson, D. E., Lock, J. E., Elde, R. P. & Thompson, T. R. Pulmonary neuroendocrine cells in hyaline membrane disease and bronchopulmonary dysplasia. Pediatr Res. 16, 446-454 (1982).
32. Johnson, D. E., Wobken, J. D. & Landrum, B. G. Changes in bombesin, calcitonin, and serotonin immunoreactive pulmonary neuroendocrine cells in cystic fibrosis and after prolonged mechanical ventilation. Am Rev Respir Dis 137, 123-31, (1988).
33. Meloni, F, et al. Urinary levels of bombesin-related peptides in a population sample from northern Italy: potential role in the pathogenesis of chronic obstructive pulmonary disease. Am J Med Sci 315, 258-65. (1998).
34. Aguayo, S. M. Determinants of susceptibility to cigarette smoke. Am. J. Respir. Crit. Care Med. 149, 1692-1698 (1994).
35. Aguayo, S. M. et al. Increased levels of bombesin-like peptides in the lower respiratory tract of asymptomatic cigarette smokers. J. Clin. Invest. 84, 1105-1113 (1989).
36. Aguayo, S. et al. Urinary levels of bombesin-like peptides in asymptomatic cigarette smokers: a potential risk marker for smoking-related diseases. Cancer Res. (Suppl.) 52, 2727s-31s (1992).
37, Gilliland, F. D. et al. Regular smoking and asthma incidence in adolescents. Am J Respir Crit. Care Med 174, 1094-100. Epub 2006 Sep. 14. (2006).
38. Aguayo, S. M. et al. Brief Report: Idiopathic diffuse hyperplasia of pulmonary neuroendocrine cells and airways disease. N. Engl. J. Med. 327, 1285-1288 (1992).
39. Deterding, R. R., Pye, C., Fan, L. L. & Langston, C. Persistent tachypnea of infancy is associated with neuroendocrine cell hyperplasia. Pediatr Pulmonol 40, 157-65. (2005).
40. Erspamer, V. Discovery, isolation, and characterization of bombesin-like peptides. Ann N Y Acad Sci 547, 3-9. (1988).
41. Sunday, M. E. Cell-specific localization of neuropeptide gene expression: Gastrin-releasing peptide (GRP; mammalian bombesin). Methods in Neurosciences 5, 123-136 (1991).
42. King, K. A., Torday, J. S. & Sunday, M. E. Bombesin and [leu8]phyllolitorin promote fetal mouse lung branching morphogenesis via a specific receptor-mediated mechanism. Proc Natl Acad Sci USA 92, 4357-4361 (1995).
43. Sunday, M. E. & Cutz, E. Role of neuroendocrine cells in fetal and postnatal lung. in Endocrinology of the Lung (ed. Mendelson, C. R.) 299-336 (Humana Press, Totowa, N.J., 2000).
44. Chang, L. et al. A catalytic antioxidant attenuates alveolar structural remodeling in bronchopulmonary dysplasia. Am. J. Respir. Cell Mol. Biol, 167, 57-64 (2003).
45. De la Fuente, M., Del Rio, M., Ferrandez, M. D. & Hernanz, A. Modulation of phagocytic function in murine peritoneal macrophages by bombesin, gastrin-releasing peptide and neuromedin C. Immunology 73, 205-11, (1991).
46. Pascual, R. M. & Peters, S. P. Airway remodeling contributes to the progressive loss of lung function in asthma: an overview. J Allergy Clin Immunol 116, 477-86; quiz 487. (2005).
47. Sunday, M. E. et al. Calcitonin driven v-Ha-ras induces multilineage pulmonary epithelial hyperplasias and neoplasms. Oncogene 18, 4336-4347 (1999).
48. Joad, J. P., Ji, C., Kott, K. S., Bric, J. M. & Pinkerton, K. E. In utero and postnatal effects of sidestream cigarette smoke exposure on lung function, hyperresponsiveness, and neuroendocrine cells in rats. Toxicol Appl Pharmacol 132, 63-71. (1995).
49. Li, K., Nagalla, S. R. & Spindel, E. R. A rhesus monkey model to characterize the role of gastrin-releasing peptide (GRP) in lung development. J. Clin. Invest. 94, 1605-1615 (1994).
50. Makino, S. et al. Definition, diagnosis, disease types, and classification of asthma. Int Arch Allergy Immunol 136, 3-4. (2005).
51. Sontag, S. J. The spectrum of pulmonary symptoms due to gastroesophageal reflux. Thorac Surg Clin 15, 353-68. (2005).
52. Joad, J. P. et al. Structural and functional localization of airway effects from episodic exposure of infant monkeys to allergen and/or ozone. Toxicol Appl Pharmacol. 214:237-43 (2006).
53. Martinez, A., Zudaire, E., Julian, M., Moody, T. W. & Cuttitta, F. Gastrin-releasing peptide (GRP) induces angiogenesis and the specific GRP blocker 77427 inhibits tumor growth in vitro and in vivo. Oncogene 24, 4106-4113 (2005).
54. Martinez, A. et al. Identification of vasoactive nonpeptidic positive and negative modulators of adrenomedullin using a neutralizing antibody-based screening strategy. Endocrinol. 145, 3858-3865 (2004).
55. Cuttitta, F. et al. Bombesin-like peptides can function as autocrine growth factors in human small cell cancer. Nature 316, 823-826 (1985).

The foregoing is illustrative of the present invention, and is not to be construed as limiting thereof. The invention is defined by the following claims, with equivalents of the claims to be included therein.

1-8. (canceled)

9. A method of treating pulmonary fibrosis in a subject in need thereof, comprising administering to said subject a gastrin-releasing peptide (GRP) inhibitor in a treatment effective amount.

10. The method of claim 9, wherein said subject is afflicted with idiopathic pulmonary fibrosis, other interstitial lung diseases, or radiation pneumonitis.

11-12. (canceled)

13. The method of claim 9, wherein said administering step is carried out by topically applying said GRP inhibitor to airway surfaces of said subject.

14. The method of claim 9, wherein said administering step is carried out by inhalation administration.

15. The method of claim 9, wherein said GRP inhibitor is a small molecule GRP inhibitor.

16. The method of claim 9, wherein said GRP inhibitor is a compound of the formula: 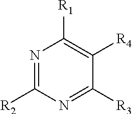
wherein:
R₁ is —R₅—(CH₂)ₙ—CH(R₆)OH, and R₅ is NH, S or O, R₆ is H or CH₃, and n is an integer from 1-4;
R₂ is NH₂, substituted amino or acetamide;
R₃ is H, halogen, CH₃, or CF₃; and
R₄ is H, alkyl, substituted alkyl, alkenyl, alkoxy or halogen;
or a pharmaceutically acceptable salt or prodrug thereof.

17. The method of claim 9, wherein said GRP inhibitor is a compound of the formula: 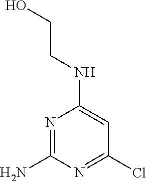 [SMILES: Nc1nc(Cl)cc(NCCO)n1]
or a pharmaceutically acceptable salt or prodrug thereof.

18. The method of claim 9, wherein said GRP inhibitor is the monoclonal antibody 2A11, or a monoclonal antibody that specifically binds to the epitope bound by the monoclonal antibody 2A11.

19-20. (canceled)
